(19) United States
(12) Patent Application Publication
(10) Pub. No.: US 2023/0013884 A1
(43) Pub. Date: Jan. 19, 2023

(54) ENDOSCOPE WITH SYNTHETIC APERTURE MULTISPECTRAL CAMERA ARRAY

(71) Applicant: Cilag GmbH International, Zug (CH)

(72) Inventors: Tarik Yardibi, Wayland, MA (US); Emir Osmanagic, Norwell, MA (US); Jihang Wang, Sewickley, PA (US); Patrick J. Treado, Pittsburgh, PA (US); Vijai Thottathil Jayadevan, Monroeville, PA (US)

(21) Appl. No.: 17/375,593

(22) Filed: Jul. 14, 2021

(51) Int. Cl. A61B 1/045; A61B 34/20; G06T 3/00; G06T 3/20; G06T 3/60; G06T 3/40; A61B 1/00

(52) U.S. Cl. CPC A61B 1/045; A61B 34/20; G06T 3/0006; G06T 3/20; G06T 3/60; G06T 3/4007; A61B 1/0005; A61B 1/00096

(57) ABSTRACT

A method which may effectively provide an endoscope or other surgical instrument with a synthetic multi-camera array may comprise capturing using one or more cameras located at a distal tip of the surgical instrument, a set of images comprising first and second images. For each image in such set of images, that image may be captured by a corresponding camera from the one or more cameras, may be captured when the distal dip of the instrument is located at a corresponding point in space. Such a method may also comprise generating a three dimensional image based on compositing representations of a structure in the first and second image after applying a non-rigid transformation to one or more of those representations.

### BACKGROUND

Surgical systems may incorporate an imaging system, which may allow the clinician(s) to view the surgical site and/or one or more portions thereof on one or more displays such as a monitor. The display(s) may be local and/or remote to a surgical theater. An imaging system may include a scope with a camera that views the surgical site and transmits the view to a display that is viewable by the clinician. Scopes include, but are not limited to, laparoscopes, robotic laparoscopes, arthroscopes, angioscopes, bronchoscopes, choledochoscopes, colonoscopes, cytoscopes, duodenoscopes, enteroscopes, esophagogastro-duodenoscopes (gastroscopes), endoscopes, laryngoscopes, nasopharyngo-neproscopes, sigmoidoscopes, thoracoscopes, ureteroscopes, and exoscopes. Imaging systems may be limited by the information that they are able to recognize and/or convey to the clinician(s). For example, certain concealed structures, physical contours, and/or dimensions within a three-dimensional space may be unrecognizable intraoperatively by certain imaging systems. Additionally, certain imaging systems may be incapable of communicating and/or conveying certain information to the clinician(s) intraoperatively.

Examples of surgical imaging systems are disclosed in U.S. Pat. Pub. No. 2020/0015925, entitled “Combination Emitter and Camera Assembly,” published Jan. 16, 2020; U.S. Pat. Pub. No. 2020/0015899, entitled “Surgical Visualization with Proximity Tracking Features,” published Jan. 16, 2020; U.S. Pat. Pub. No. 2020/0015924, entitled “Robotic Light Projection Tools,” published Jan. 16, 2020; and U.S. Pat. Pub. No. 2020/0015898, entitled “Surgical Visualization Feedback System,” published Jan. 16, 2020. The disclosure of each of the above-cited U.S. patents and patent applications is incorporated by reference herein in its entirety.

While various kinds of surgical instruments and systems have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 depicts a schematic view of an exemplary surgical visualization system including an imaging device and a surgical device;

FIG. 2 depicts a schematic diagram of an exemplary control system that may be used with the surgical visualization system of FIG. 1;

FIG. 3 depicts a schematic diagram of another exemplary control system that may be used with the surgical visualization system of FIG. 1;

FIG. 4 depicts exemplary hyperspectral identifying signatures to differentiate anatomy from obscurants, and more particularly depicts a graphical representation of a ureter signature versus obscurants;

FIG. 5 depicts exemplary hyperspectral identifying signatures to differentiate anatomy from obscurants, and more particularly depicts a graphical representation of an artery signature versus obscurants;

FIG. 6 depicts exemplary hyperspectral identifying signatures to differentiate anatomy from obscurants, and more particularly depicts a graphical representation of a nerve signature versus obscurants;

FIG. 7A depicts a schematic view of an exemplary emitter assembly that may be incorporated into the surgical visualization system of FIG. 1, the emitter assembly including a single electromagnetic radiation (EMR) source, showing the emitter assembly in a first state;

FIG. 7B depicts a schematic view of the emitter assembly of FIG. 7A, showing the emitter assembly in a second state;

FIG. 7C depicts a schematic view of the emitter assembly of FIG. 7A, showing the emitter assembly in a third state;

FIG. 8 depicts a method which may be used to combine information from multiple images;

FIG. 9 depicts a method which may be used in translating cameras used to capture images; and

FIG. 10 depicts a combination of multiple obstructed images to obtain a single unobstructed image.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical device. The term “proximal” refers to the position of an element arranged closer to the surgeon, and the term “distal” refers to the position of an element arranged further away from the surgeon. Moreover, to the extent that spatial terms such as “top,” “bottom,” “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose(s) described herein.

Similarly, the phrase “based on” should be understood as referring to a relationship in which one thing is determined at least in part by what it is specified as being “based on.” This includes, but is not limited to, relationships where one thing is exclusively determined by another, which relationships may be referred to using the phrase “exclusively based on.”

### I. Exemplary Surgical Visualization System

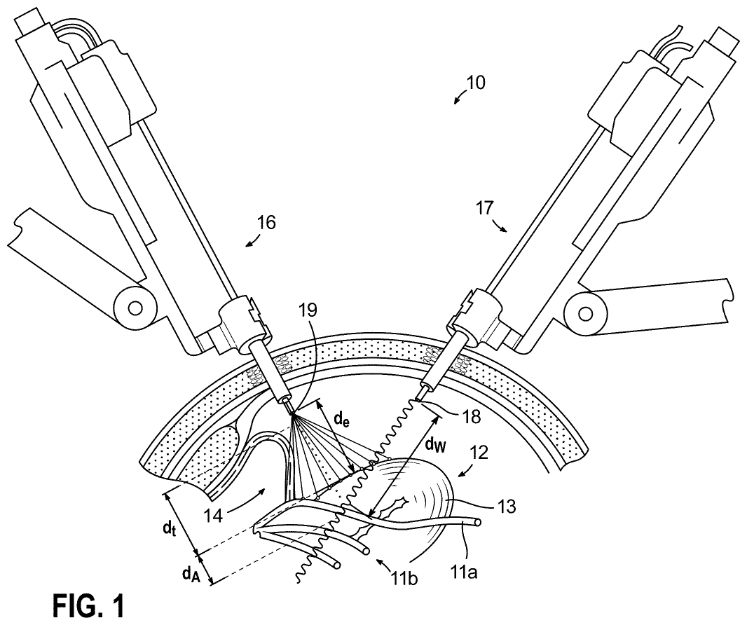

FIG. 1 depicts a schematic view of a surgical visualization system (10) according to at least one aspect of the present disclosure. The surgical visualization system (10) may create a visual representation of a critical structure (11a, 11b) within an anatomical field. The surgical visualization system (10) may be used for clinical analysis and/or medical intervention, for example. In certain instances, the surgical visualization system (10) may be used intraoperatively to provide real-time, or near real-time, information to the clinician regarding proximity data, dimensions, and/or distances during a surgical procedure. The surgical visualization system (10) is configured for intraoperative identification of critical structure(s) and/or to facilitate the avoidance of critical structure(s) (11a, 11b) by a surgical device. For example, by identifying critical structures (11a, 11b), a clinician may avoid maneuvering a surgical device into a critical structure (11a, 11b) and/or a region in a predefined proximity of a critical structure (11a, 11b) during a surgical procedure. The clinician may avoid dissection of and/or near a vein, artery, nerve, and/or vessel, for example, identified as a critical structure (11a, 11b), for example. In various instances, critical structure(s) (11a, 11b) may be determined on a patient-by-patient and/or a procedure-by-procedure basis.

Critical structures (11a, 11b) may be any anatomical structures of interest. For example, a critical structure (11a, 11b) may be a ureter, an artery such as a superior mesenteric artery, a vein such as a portal vein, a nerve such as a phrenic nerve, and/or a sub-surface tumor or cyst, among other anatomical structures. In other instances, a critical structure (11a, 11b) may be any foreign structure in the anatomical field, such as a surgical device, surgical fastener, clip, tack, bougie, band, and/or plate, for example. In one aspect, a critical structure (11a, 11b) may be embedded in tissue. Stated differently, a critical structure (11a, 11b) may be positioned below a surface of the tissue. In such instances, the tissue conceals the critical structure (11a, 11b) from the clinician's view. A critical structure (11a, 11b) may also be obscured from the view of an imaging device by the tissue. The tissue may be fat, connective tissue, adhesions, and/or organs, for example. In other instances, a critical structure (11a, 11b) may be partially obscured from view. A surgical visualization system (10) is shown being utilized intraoperatively to identify and facilitate avoidance of certain critical structures, such as a ureter (11a) and vessels (11b) in an organ (12) (the uterus in this example), that are not visible on a surface (13) of the organ (12).

A. Overview of Exemplary Surgical Visualization System

With continuing reference to FIG. 1, the surgical visualization system (10) incorporates tissue identification and geometric surface mapping in combination with a distance sensor system (14). In combination, these features of the surgical visualization system (10) may determine a position of a critical structure (11a, 11b) within the anatomical field and/or the proximity of a surgical device (16) to the surface (13) of the visible tissue and/or to a critical structure (11a, 11b). The surgical device (16) may include an end effector having opposing jaws (not shown) and/or other structures extending from the distal end of the shaft of the surgical device (16). The surgical device (16) may be any suitable surgical device such as, for example, a dissector, a stapler, a grasper, a clip applier, a monopolar RF electrosurgical instrument, a bipolar RF electrosurgical instrument, and/or an ultrasonic instrument. As described herein, a surgical visualization system (10) may be configured to achieve identification of one or more critical structures (11a, 11b) and/or the proximity of a surgical device (16) to critical structure(s) (11a, 11b).

The depicted surgical visualization system (10) includes an imaging system that includes an imaging device (17), such as a camera of a scope, for example, that is configured to provide real-time views of the surgical site. In various instances, an imaging device (17) includes a spectral camera (e.g., a hyperspectral camera, multispectral camera, a fluorescence detecting camera, or selective spectral camera), which is configured to detect reflected or emitted spectral waveforms and generate a spectral cube of images based on the molecular response to the different wavelengths. Views from the imaging device (17) may be provided to a clinician; and, in various aspects of the present disclosure, may be augmented with additional information based on the tissue identification, landscape mapping, and input from a distance sensor system (14). In such instances, a surgical visualization system (10) includes a plurality of subsystems—an imaging subsystem, a surface mapping subsystem, a tissue identification subsystem, and/or a distance determining subsystem. These subsystems may cooperate to intraoperatively provide advanced data synthesis and integrated information to the clinician(s).

The imaging device (17) of the present example includes an emitter (18), which is configured to emit spectral light in a plurality of wavelengths to obtain a spectral image of hidden structures, for example. The imaging device (17) may also include a three-dimensional camera and associated electronic processing circuits in various instances. In one aspect, the emitter (18) is an optical waveform emitter that is configured to emit electromagnetic radiation (e.g., near-infrared radiation (NIR) photons) that may penetrate the surface (13) of a tissue (12) and reach critical structure(s) (11a, 11b). The imaging device (17) and optical waveform emitter (18) thereon may be positionable by a robotic arm or a surgeon manually operating the imaging device. A corresponding waveform sensor (e.g., an image sensor, spectrometer, or vibrational sensor, etc.) on the imaging device (17) may be configured to detect the effect of the electromagnetic radiation received by the waveform sensor.

The wavelengths of the electromagnetic radiation emitted by the optical waveform emitter (18) may be configured to enable the identification of the type of anatomical and/or physical structure, such as critical structure(s) (11a, 11b). The identification of critical structure(s) (11a, 11b) may be accomplished through spectral analysis, photo-acoustics, fluorescence detection, and/or ultrasound, for example. In one aspect, the wavelengths of the electromagnetic radiation may be variable. The waveform sensor and optical waveform emitter (18) may be inclusive of a multispectral imaging system and/or a selective spectral imaging system, for example. In other instances, the waveform sensor and optical waveform emitter (18) may be inclusive of a photoacoustic imaging system, for example. In other instances, an optical waveform emitter (18) may be positioned on a separate surgical device from the imaging device (17). By way of example only, the imaging device (17) may provide hyperspectral imaging in accordance with at least some of the teachings of U.S. Pat. No. 9,274,047, entitled “System and Method for Gross Anatomic Pathology Using Hyperspectral Imaging,” issued Mar. 1, 2016, the disclosure of which is incorporated by reference herein in its entirety.

The depicted surgical visualization system (10) also includes an emitter (19), which is configured to emit a pattern of light, such as stripes, grid lines, and/or dots, to enable the determination of the topography or landscape of a surface (13). For example, projected light arrays may be used for three-dimensional scanning and registration on a surface (13). The projected light arrays may be emitted from an emitter (19) located on a surgical device (16) and/or an imaging device (17), for example. In one aspect, the projected light array is employed to determine the shape defined by the surface (13) of the tissue (12) and/or the motion of the surface (13) intraoperatively. An imaging device (17) is configured to detect the projected light arrays reflected from the surface (13) to determine the topography of the surface (13) and various distances with respect to the surface (13). By way of further example only, a visualization system (10) may utilize patterned light in accordance with at least some of the teachings of U.S. Pat. Pub. No. 2017/0055819, entitled “Set Comprising a Surgical Instrument,” published Mar. 2, 2017, the disclosure of which is incorporated by reference herein in its entirety; and/or U.S. Pat. Pub. No. 2017/0251900, entitled “Depiction System,” published Sep. 7, 2017, the disclosure of which is incorporated by reference herein in its entirety.

The depicted surgical visualization system (10) also includes a distance sensor system (14) configured to determine one or more distances at the surgical site. In one aspect, the distance sensor system (14) may include a time-of-flight distance sensor system that includes an emitter, such as the structured light emitter (19); and a receiver (not shown), which may be positioned on the surgical device (16). In other instances, the time-of-flight emitter may be separate from the structured light emitter (19). In one general aspect, the emitter portion of the time-of-flight distance sensor system (14) may include a laser source and the receiver portion of the time-of-flight distance sensor system (14) may include a matching sensor. A time-of-flight distance sensor system (14) may detect the “time of flight,” or how long the laser light emitted by the structured light emitter (19) has taken to bounce back to the sensor portion of the receiver. Use of a very narrow light source in a structured light emitter (19) may enable a distance sensor system (14) to determine the distance to the surface (13) of the tissue (12) directly in front of the distance sensor system (14).

Referring still to FIG. 1, a distance sensor system (14) may be employed to determine an emitter-to-tissue distance (dₑ) from a structured light emitter (19) to the surface (13) of the tissue (12). A device-to-tissue distance (dₜ) from the distal end of the surgical device (16) to the surface (13) of the tissue (12) may be obtainable from the known position of the emitter (19) on the shaft of the surgical device (16) relative to the distal end of the surgical device (16). In other words, when the distance between the emitter (19) and the distal end of the surgical device (16) is known, the device-to-tissue distance (dₜ) may be determined from the emitter-to-tissue distance (dₑ). In certain instances, the shaft of a surgical device (16) may include one or more articulation joints; and may be articulatable with respect to the emitter (19) and the jaws. The articulation configuration may include a multi-joint vertebrae-like structure, for example. In certain instances, a three-dimensional camera may be utilized to triangulate one or more distances to the surface (13).

As described above, a surgical visualization system (10) may be configured to determine the emitter-to-tissue distance (dₑ) from an emitter (19) on a surgical device (16) to the surface (13) of a uterus (12) via structured light. The surgical visualization system (10) is configured to extrapolate a device-to-tissue distance (dₜ) from the surgical device (16) to the surface (13) of the uterus (12) based on emitter-to-tissue distance (dₑ). The surgical visualization system (10) is also configured to determine a tissue-to-ureter distance (d_{A}) from a ureter (11a) to the surface (13) and a camera-to-ureter distance (d_{w}), from the imaging device (17) to the ureter (11a). Surgical visualization system (10) may determine the camera-to-ureter distance (d_{w}), with spectral imaging and time-of-flight sensors, for example. In various instances, a surgical visualization system (10) may determine (e.g., triangulate) a tissue-to-ureter distance (d_{A}) (or depth) based on other distances and/or the surface mapping logic described herein.

B. First Exemplary Control System

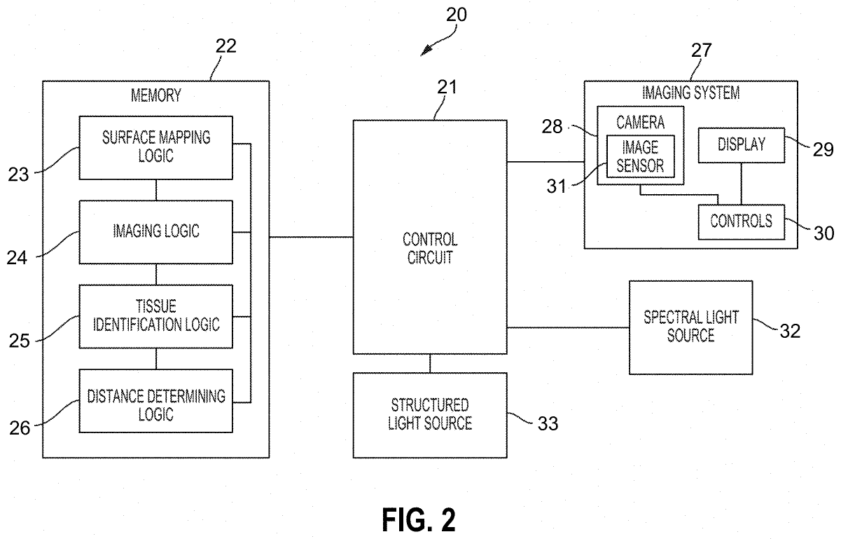

FIG. 2 is a schematic diagram of a control system (20), which may be utilized with a surgical visualization system (10). The depicted control system (20) includes a control circuit (21) in signal communication with a memory (22). The memory (22) stores instructions executable by the control circuit (21) to determine and/or recognize critical structures (e.g., critical structures (11a, 11b) depicted in FIG. 1), determine and/or compute one or more distances and/or three-dimensional digital representations, and to communicate certain information to one or more clinicians. For example, a memory (22) stores surface mapping logic (23), imaging logic (24), tissue identification logic (25), or distance determining logic (26) or any combinations of logic (23, 24, 25, 26). The control system (20) also includes an imaging system (27) having one or more cameras (28) (like the imaging device (17) depicted in FIG. 1), one or more displays (29), one or more controls (30) or any combinations of these elements. The one or more cameras (28) may include one or more image sensors (31) to receive signals from various light sources emitting light at various visible and invisible spectra (e.g., visible light, spectral imagers, three-dimensional lens, among others). The display (29) may include one or more screens or monitors for depicting real, virtual, and/or virtually-augmented images and/or information to one or more clinicians.

In various aspects, a main component of a camera (28) includes an image sensor (31). An image sensor (31) may include a Charge-Coupled Device (CCD) sensor, a Complementary Metal Oxide Semiconductor (CMOS) sensor, a short-wave infrared (SWIR) sensor, a hybrid CCD/CMOS architecture (sCMOS) sensor, and/or any other suitable kind(s) of technology. An image sensor (31) may also include any suitable number of chips.

The depicted control system (20) also includes a spectral light source (32) and a structured light source (33). In certain instances, a single source may be pulsed to emit wavelengths of light in the spectral light source (32) range and wavelengths of light in the structured light source (33) range. Alternatively, a single light source may be pulsed to provide light in the invisible spectrum (e.g., infrared spectral light) and wavelengths of light on the visible spectrum. A spectral light source (32) may include a hyperspectral light source, a multispectral light source, a fluorescence excitation light source, and/or a selective spectral light source, for example. In various instances, tissue identification logic (25) may identify critical structure(s) via data from a spectral light source (32) received by the image sensor (31) portion of a camera (28). Surface mapping logic (23) may determine the surface contours of the visible tissue based on reflected structured light. With time-of-flight measurements, distance determining logic (26) may determine one or more distance(s) to the visible tissue and/or critical structure(s) (11a, 11b). One or more outputs from surface mapping logic (23), tissue identification logic (25), and distance determining logic (26), may be provided to imaging logic (24), and combined, blended, and/or overlaid to be conveyed to a clinician via the display (29) of the imaging system (27).

C. Second Exemplary Control System

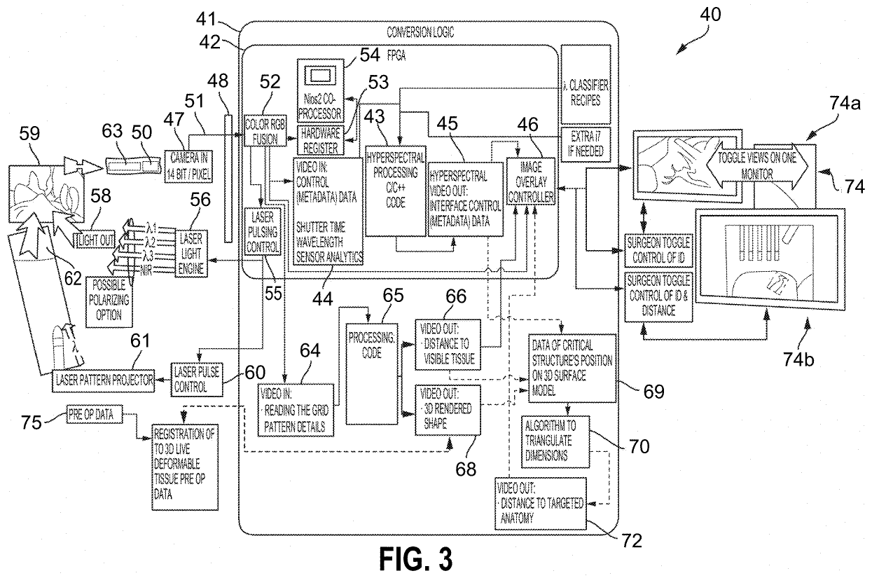

FIG. 3 depicts a schematic of another control system (40) for a surgical visualization system, such as the surgical visualization system (10) depicted in FIG. 1, for example. This control system (40) is a conversion system that integrates spectral signature tissue identification and structured light tissue positioning to identify critical structures, especially when those structures are obscured by other tissue, such as fat, connective tissue, blood, and/or other organs, for example. Such technology could also be useful for detecting tissue variability, such as differentiating tumors and/or non-healthy tissue from healthy tissue within an organ.

The control system (40) depicted in FIG. 3 is configured for implementing a hyperspectral or fluorescence imaging and visualization system in which a molecular response is utilized to detect and identify anatomy in a surgical field of view. This control system (40) includes a conversion logic circuit (41) to convert tissue data to surgeon usable information. For example, the variable reflectance based on wavelengths with respect to obscuring material may be utilized to identify a critical structure in the anatomy. Moreover, this control system (40) combines the identified spectral signature and the structured light data in an image. For example, this control system (40) may be employed to create a three-dimensional data set for surgical use in a system with augmentation image overlays. Techniques may be employed both intraoperatively and preoperatively using additional visual information. In various instances, this control system (40) is configured to provide warnings to a clinician when in the proximity of one or more critical structures. Various algorithms may be employed to guide robotic automation and semi-automated approaches based on the surgical procedure and proximity to the critical structure(s).

The control system (40) depicted in FIG. 3 is configured to detect the critical structure(s) and provide an image overlay of the critical structure and measure the distance to the surface of the visible tissue and the distance to the embedded/buried critical structure(s). In other instances, this control system (40) may measure the distance to the surface of the visible tissue or detect the critical structure(s) and provide an image overlay of the critical structure.

The control system (40) depicted in FIG. 3 includes a spectral control circuit (42). The spectral control circuit (42) includes a processor (43) to receive video input signals from a video input processor (44). The processor (43) is configured to process the video input signal from the video input processor (44) and provide a video output signal to a video output processor (45), which includes a hyperspectral video-out of interface control (metadata) data, for example. The video output processor (45) provides the video output signal to an image overlay controller (46).

The video input processor (44) is coupled to a camera (47) at the patient side via a patient isolation circuit (48). As previously discussed, the camera (47) includes a solid state image sensor (50). The camera (47) receives intraoperative images through optics (63) and the image sensor (50). An isolated camera output signal (51) is provided to a color RGB fusion circuit (52), which employs a hardware register (53) and a Nios2 co-processor (54) to process the camera output signal (51). A color RGB fusion output signal is provided to the video input processor (44) and a laser pulsing control circuit (55).

The laser pulsing control circuit (55) controls laser light engine (56). In some versions, light engine (56) includes any one or more of lasers, LEDs, incandescent sources, and/or interface electronics configured to illuminate the patient's body habitus with a chosen light source for imaging by a camera and/or analysis by a processor. The light engine (56) outputs light in a plurality of wavelengths (λ1, λ2, λ3 . . . λn) including near infrared (NIR) and broadband white light. The light output (58) from the light engine (56) illuminates targeted anatomy in an intraoperative surgical site (59). The laser pulsing control circuit (55) also controls a laser pulse controller (60) for a laser pattern projector (61) that projects a laser light pattern (62), such as a grid or pattern of lines and/or dots, at a predetermined wavelength (λ2) on the operative tissue or organ at the surgical site (59). The camera (47) receives the patterned light as well as the reflected or emitted light output through camera optics (63). The image sensor (50) converts the received light into a digital signal.

The color RGB fusion circuit (52) also outputs signals to the image overlay controller (46) and a video input module (64) for reading the laser light pattern (62) projected onto the targeted anatomy at the surgical site (59) by the laser pattern projector (61). A processing module (65) processes the laser light pattern (62) and outputs a first video output signal (66) representative of the distance to the visible tissue at the surgical site (59). The data is provided to the image overlay controller (46). The processing module (65) also outputs a second video signal (68) representative of a three-dimensional rendered shape of the tissue or organ of the targeted anatomy at the surgical site.

The first and second video output signals (66, 68) include data representative of the position of the critical structure on a three-dimensional surface model, which is provided to an integration module (69). In combination with data from the video output processor (45) of the spectral control circuit (42), the integration module (69) may determine distance (d_{A}) (FIG. 1) to a buried critical structure (e.g., via triangularization algorithms (70)), and that distance (d_{A}) may be provided to the image overlay controller (46) via a video out processor (72). The foregoing conversion logic may encompass a conversion logic circuit (41), intermediate video monitors (74), and a camera (56)/laser pattern projector (61) positioned at surgical site (59).

Preoperative data (75) from a CT or MRI scan may be employed to register or align certain three-dimensional deformable tissue in various instances. Such preoperative data (75) may be provided to an integration module (69) and ultimately to the image overlay controller (46) so that such information may be overlaid with the views from the camera (47) and provided to video monitors (74). Registration of preoperative data is further described herein and in U.S. Pat. Pub. No. 2020/0015907, entitled “Integration of Imaging Data,” published Jan. 16, 2020, for example, which is incorporated by reference herein in its entirety.

Video monitors (74) may output the integrated/augmented views from the image overlay controller (46). On a first monitor (74a), the clinician may toggle between (A) a view in which a three-dimensional rendering of the visible tissue is depicted and (B) an augmented view in which one or more hidden critical structures are depicted over the three-dimensional rendering of the visible tissue. On a second monitor (74b), the clinician may toggle on distance measurements to one or more hidden critical structures and/or the surface of visible tissue, for example.

D. Exemplary Hyperspectral Identifying Signatures

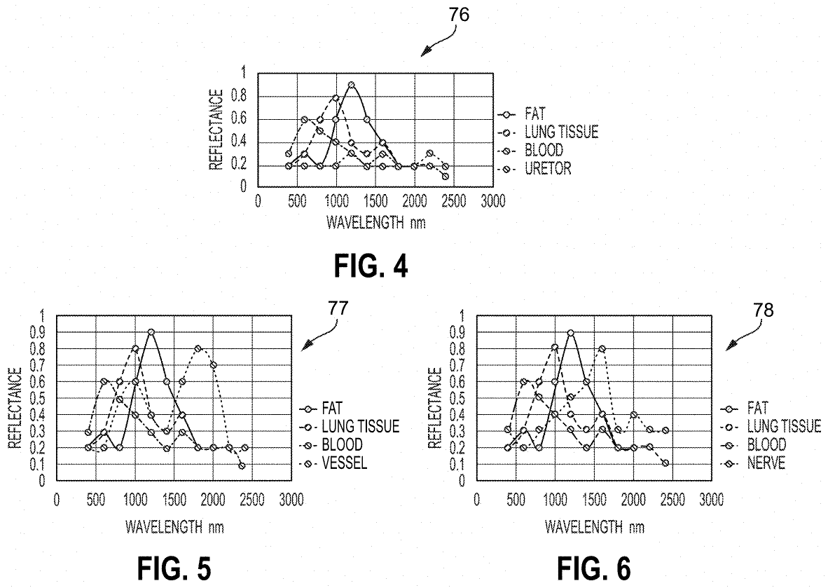

FIG. 4 depicts a graphical representation (76) of an illustrative ureter signature versus obscurants. The plots represent reflectance as a function of wavelength (nm) for wavelengths for fat, lung tissue, blood, and a ureter. FIG. 5 depicts a graphical representation (77) of an illustrative artery signature versus obscurants. The plots represent reflectance as a function of wavelength (nm) for fat, lung tissue, blood, and a vessel. FIG. 6 depicts a graphical representation (78) of an illustrative nerve signature versus obscurants. The plots represent reflectance as a function of wavelength (nm) for fat, lung tissue, blood, and a nerve.

In various instances, select wavelengths for spectral imaging may be identified and utilized based on the anticipated critical structures and/or obscurants at a surgical site (i.e., “selective spectral” imaging). By utilizing selective spectral imaging, the amount of time required to obtain the spectral image may be minimized such that the information may be obtained in real-time, or near real-time, and utilized intraoperatively. In various instances, the wavelengths may be selected by a clinician or by a control circuit based on input by the clinician. In certain instances, the wavelengths may be selected based on machine learning and/or big data accessible to the control circuit via a cloud, for example.

E. Exemplary Singular EMR Source Emitter Assembly

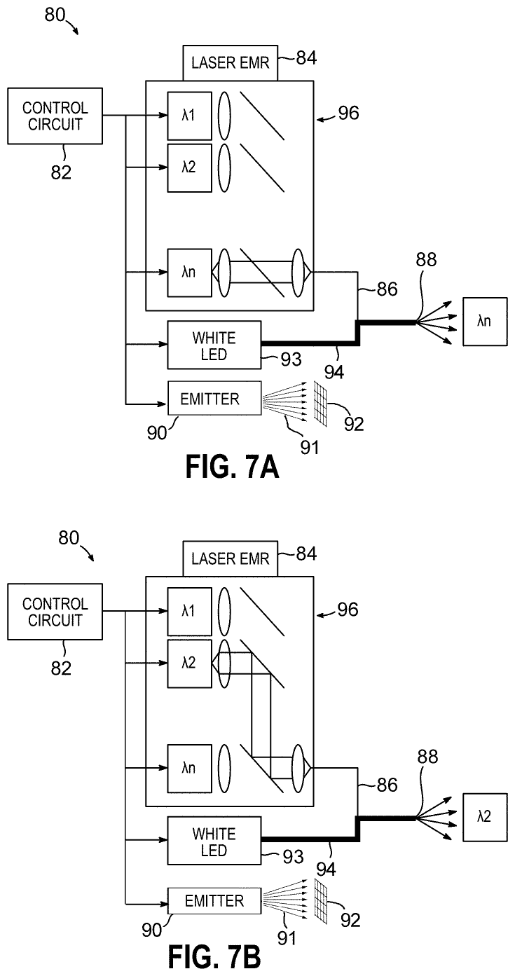

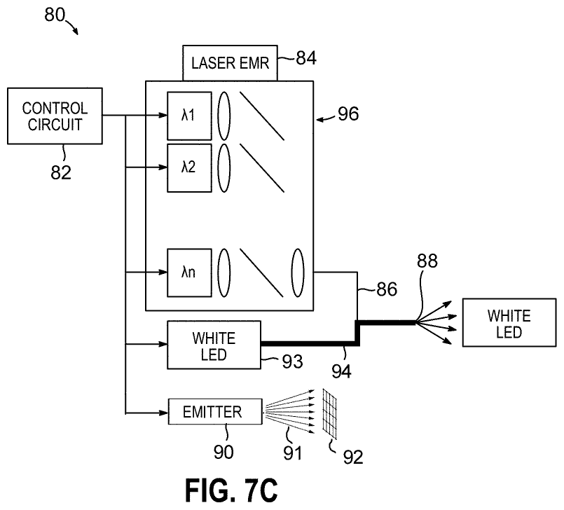

Referring now to FIGS. 7A-7C, in one aspect, a visualization system (10) includes a receiver assembly (e.g., positioned on a surgical device (16)), which may include a camera (47) including an image sensor (50) (FIG. 3), and an emitter assembly (80) (e.g., positioned on imaging device (17)), which may include an emitter (18) (FIG. 1) and/or a laser light engine (56) (FIG. 3). Further, a visualization system (10) may include a control circuit (82), which may include the control circuit (21) depicted in FIG. 2 and/or the spectral control circuit (42) depicted in FIG. 3, coupled to each of emitter assembly (80) and the receiver assembly. An emitter assembly (80) may be configured to emit EMR at a variety of wavelengths (e.g., in the visible spectrum and/or in the IR spectrum) and/or as structured light (i.e., EMR projected in a particular known pattern as described below). A control circuit (82) may include, for example, hardwired circuitry, programmable circuitry (e.g., a computer processor coupled to a memory or field programmable gate array), state machine circuitry, firmware storing instructions executed by programmable circuitry, and any combination thereof.

In one aspect, an emitter assembly (80) may be configured to emit visible light, IR, and/or structured light from a single EMR source (84). For example, FIGS. 7A-7C illustrate a diagram of the emitter assembly (80) in alternative states, in accordance with at least one aspect of the present disclosure. In this aspect, the emitter assembly (80) comprises a channel (86) connecting an EMR source (84) to a first emitter (88) configured to emit visible light (e.g., RGB), IR. The channel (86) may include, for example, a fiber optic cable. The EMR source (84) may include, for example, a light engine (56) (FIG. 3) including a plurality of light sources configured to selectively output light at respective wavelengths. In the example shown, the emitter assembly (80) comprises a white LED (93) connected to the first emitter (88) via another channel (94). A second emitter (90) is configured to emit structured light (91) in response to being supplied EMR of particular wavelengths from the EMR source (84). The second emitter (90) may include a filter configured to emit EMR from the EMR source (84) as structured light (91) to cause the emitter assembly (80) to project a predetermined pattern (92) onto the target site.

The depicted emitter assembly (80) further includes a wavelength selector assembly (96) configured to direct EMR emitted from the light sources of the EMR source (84) toward the first emitter (88). In the depicted aspect, the wavelength selector assembly (96) includes a plurality of deflectors and/or reflectors configured to transmit EMR from the light sources of the EMR source (84).

In one aspect, a control circuit (82) may be electrically coupled to each light source of the EMR source (84) such that it may control the light outputted therefrom via applying voltages or control signals thereto. The control circuit (82) may be configured to control the light sources of the EMR source (84) to direct EMR from the EMR source (84) to the first emitter (88) in response to, for example, user input and/or detected parameters (e.g., parameters associated with the surgical instrument or the surgical site). In one aspect, the control circuit (82) is coupled to the EMR source (84) such that it may control the wavelength of the EMR generated by the EMR source (84). In various aspects, the control circuit (82) may control the light sources of the EMR source (84) either independently or in tandem with each other.

In some aspects, the control circuit (82) may adjust the wavelength of the EMR generated by the EMR source (84) according to which light sources of the EMR source (84) are activated. In other words, the control circuit (82) may control the EMR source (84) so that it produces EMR at a particular wavelength or within a particular wavelength range. For example, in FIG. 7A, the control circuit (82) has applied control signals to the nth light source of the EMR source (84) to cause it to emit EMR at an nth wavelength (λn), and has applied control signals to the remaining light sources of the EMR source (84) to prevent them from emitting EMR at their respective wavelengths. Conversely, in FIG. 7B the control circuit (82) has applied control signals to the second light source of the EMR source (84) to cause it to emit EMR at a second wavelength (λ2), and has applied control signals to the remaining light sources of the EMR source (84) to prevent them from emitting EMR at their respective wavelengths. Furthermore, in FIG. 7C the control circuit (82) has applied control signals to the light sources of the EMR source (84) to prevent them from emitting EMR at their respective wavelengths, and has applied control signals to a white LED source to cause it to emit white light.

In addition to the foregoing, at least part of any one or more of the surgical visualization system (10) depicted in FIG. 1, the control system (20) depicted in FIG. 2, the control system (40) depicted in FIG. 3, and/or the emitter assembly (80) depicted in FIGS. 7A and 7B may be configured and operable in accordance with at least some of the teachings of U.S. Pat. Pub. No. 2020/0015925, entitled “Combination Emitter and Camera Assembly,” published Jan. 16, 2020, which is incorporated by reference above. In one aspect, a surgical visualization system (10) may be incorporated into a robotic system in accordance with at least some of such teachings.

### II. Exemplary Synthetic Aperture Multi Spectral Surgical Visualization

In some instances, it may be desirable for a surgical visualization system to combine image data captured at multiple positions and/or times to provide a richer picture of a surgical site. For example, by moving a camera around the surgical site and capturing images of the site from different perspectives, it may be possible to, essentially, create a synthetic camera with a much larger baseline (i.e., distance between stereo imaging elements) than would be possible by simply providing multiple cameras on any one instrument, thereby allowing generation of a much higher resolution three dimensional map of the surgical site than may otherwise be possible. Similarly, as a result of inhomogeneity in obscurations, not all regions of a target structure may be visible in all images, and so identifying and combining multiple images which have good visibility in certain regions may allow a synthesized image to be created which provides an improved view of the entire target structure or other aspects of the surgical site.

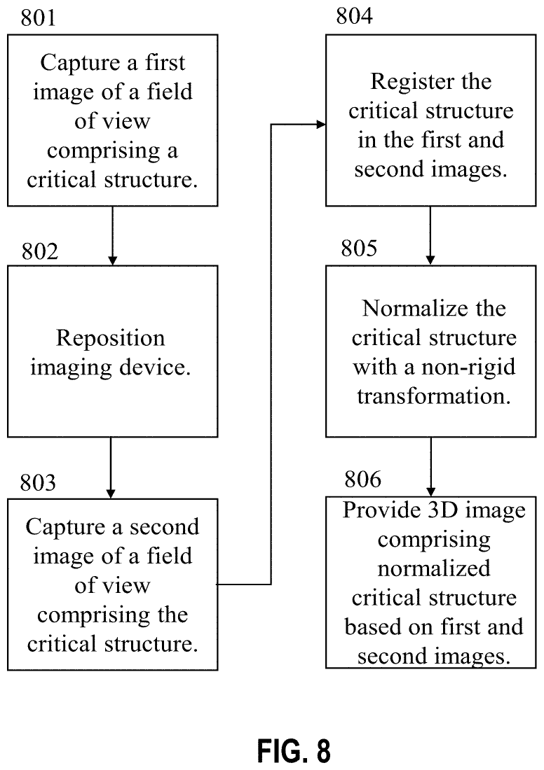

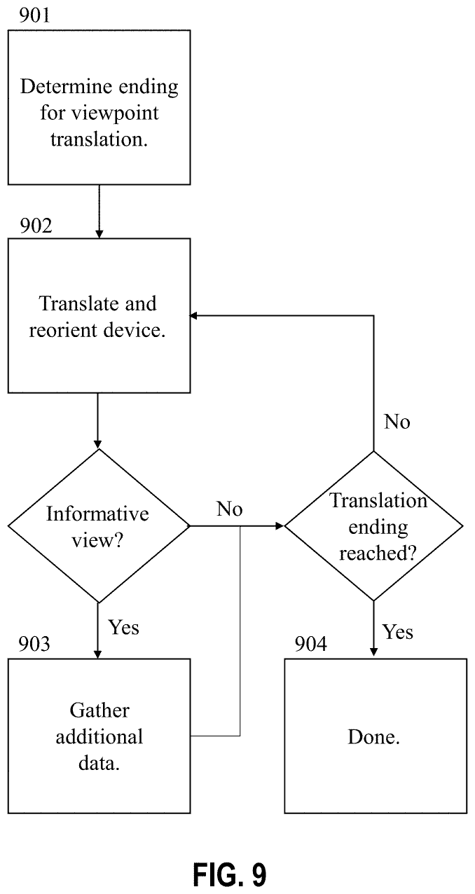

To facilitate the combination of multiple images, some implementations may augment a surgical visualization system (10) with features allowing the position and orientation of one or more cameras located at the distal tip of an instrument (e.g., an endoscope) to be tracked. For example, an endoscope or other device may be instrumented with an inertial measurement unit (IMU) and data from that IMU may be communicated back to the control circuit (21) to enable it to determine the position and orientation of the camera(s) located at the device's distal tip. Alternatively, in implementations where a device is controlled robotically, a robot motion control system may track the device's position and orientation and relay that back to the control circuit rather than requiring the control circuit to derive the position and orientation from data such as could be provided by an IMU. Other approaches to tracking position and orientation, such as through use of electromagnetic sensors as described in U.S. Pat. Pub. No. 2020/0125236, entitled “Method for Real Time Update of Fly-Through Camera Placement”, published Apr. 23, 2020, the disclosure of which is hereby incorporated by reference herein in its entirety, may also be used, and so the specific types of position and orientation tracking described should be understood as being exemplary only, and should not be treated as implying limitations on the protection provided by this document or any related document.

Where they are available, position and orientation information for an endoscope can be used to combine information from multiple images using a method such as shown in FIG. 8. Initially, in the method of FIG. 8, a first image is captured (801) by a camera having a critical structure in its field of view. The camera is then repositioned (802) so that a second image can be captured providing a different perspective on the critical structure. This repositioning (802) may be achieved in a variety of manners. For example, in some cases, the repositioning may be any type of motion which happens during the course of a procedure—e.g., random motion of a manually operated endoscope. However, in some implementations, more structured movement may also be possible. An example of this type of more structured movement is provided in FIG. 9, discussed below.

FIG. 9 depicts a method which can be used to optimize motion of a surgical device with respect to obtaining imaging data. Initially, in the method of FIG. 9, a translation ending would be determined (901). This may be done, for example, by determining a most distant position within the surgical area from which a target structure that is within an imaging device's current field of view could be viewed. Alternatively, in some cases, the determination may be made by identifying a viewpoint from which there was a high probability that a view of the target structure could be obtained that was not obscured by some kind of interfering structure (e.g., fat), such as by analyzing CT or other pre-operative images of the surgical field. As another alternative, the determination may be based on finding a location from which the target structure could be imaged from a predefined angle (e.g., a 45 degree angle) relative to the line of sight between the critical structure and the then current position of the surgical device. Preferably, however the determination is performed, its performance will incorporate constraints such as the mobility and flexibility of the surgical device, and the size and shape of the surgical area, thereby ensuring that the translation ending is a point which could be reached without compromising the safety of the patient.

After the translation ending point had been determined (901), the method of FIG. 9 would continue with translating and reorienting (902) the device. This may be done, for example, by providing instructions to a robotic motion control system (or, where the device is manually controlled, to an interface accessible by the person controlling the device) to move the device closer to the translation ending while reorienting its tip so that the critical structure would remain in the field of view as the device moved.

In the method of FIG. 9 if, during the movement of the device, a position was reached from which particularly informative data could be gathered, an additional command may be sent to gather additional data (903) rather than continuing with movement to the translation ending. This may be achieved in a variety of ways. For example, in some implementations, images may continuously be captured during a surgical device's translation, and those images may be analyzed to determine if they provide relatively better information about a target structure than others. For instance, if the target structure is obscured by a substance such as fat, then image recognition routines could be applied to the images in real time as they are collected, and if the image recognition routines identify data that is more characteristic of the target structure than the obscurant (e.g., if they identify sharp edges, or long, narrow structures, which would be more characteristic of a critical structure like a nerve or ureter than an obscurant like fat), the location of the device when the image was captured could be treated as particularly informative. The user (or robotic control system, in cases where it is present), may then be instructed to take some action other than continuing to move to the translation target (e.g., pausing motion so that images with higher exposure time can be captured, capturing additional images from the vicinity of the position identified as providing particularly informative data, etc.) so that additional data could be gathered (903). This type of image capture and analysis may be performed repeatedly until the device reaches the previously determined translation ending, at which point the process could complete (904) and the image(s) gathered could be utilized in a process like that depicted in FIG. 8.

Retuning now to the process of FIG. 8, after the surgical device had been repositioned (802), whether by a process such as illustrated in FIG. 9 or otherwise, a second image could be captured (803) of the critical structure in the device's then current field of view. The critical structure could then be registered (804) in the first and second images, such as by using the position and orientation information that had been tracked during the device's movement to create a linear map between the first and second images. Additionally, to account for potential changes in the depiction of the critical structure between the first and second images, such as may be caused by breathing or other motion of the patient, the depiction of the critical structure in either the first or second image may be normalized (805) so that the depictions match each other. As with the other steps of FIG. 8, this normalization may be performed in a variety of manners. For example, a diffusion modeling process such as Demon's algorithm, or other non-rigid registration algorithms described in Xavier Pennec, Pascal Cachier and Nicholas Ayache, Understanding the “Demon's Algorithm”: 3D Non-Rigid registration by Gradient Descent, 2^{nd} Int. Conf. on Medial Image Computing and Computer-Assisted Intervention (MICCAI'99) Pages 597-605, Cambridge, UK—Sep. 19-22, 1999, the disclosure of which is hereby incorporated by reference in its entirety, may be utilized. Alternatively, the depictions of the critical structure in the first and second images may be matched using a thin-plate spline non-rigid transformation (e.g., used for measuring 18 parameters between images). Other types of non-rigid transformations may also be applied, and so the specific algorithms mentioned should be understood as being illustrative only, and should not be treated as limiting.

Finally, in FIG. 8, after the non-rigid transformation had been applied to one or more of the depictions of the critical structure, the information from the first and second images could be combined to provide (806) (e.g., on an interface presented on the display (29) of the imaging system depicted in FIG. 2) a finalized image depicting the critical structure with greater 3D resolution and/or unobscured detail than would be available in either of the first or second images when considered on their own. Additionally, in some cases, the greater information that could be derived from the combined image may be used to perform further processing on the combined image (or other images of the critical structure) to allow for additional information to be provided. For example, the better understanding of the location of a camera relative to the critical structure or other relevant anatomy may be used to optimize the parameters of a hyperspectral imaging algorithm, thereby enabling more precise molecular chemical imaging. One example here is to use the camera position to estimate the target size. Knowing the target size will help optimizing the object size parameter for the multiscale vessel enhancement filter, which is a filter to enhance tube-like structures in the current algorithm pipeline. (e.g., as described in Frangi, A. F., Niessen, W. J., Vincken, K. L., & Viergever, M. A. (1998, October). Multiscale vessel enhancement filtering, In International conference on medical image computing and computer-assisted intervention (pp. 130-137). Springer, Berlin, Heidelberg), the disclosure of which is hereby incorporated by reference in its entirety.

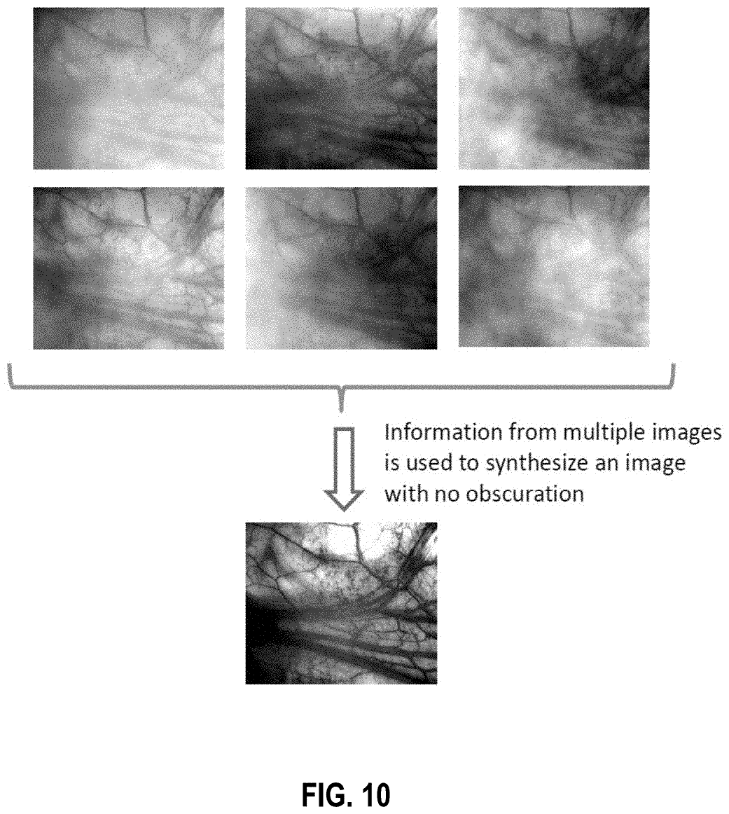

Variations beyond performing additional processing based on the more complete information available in a combined image may also be possible in some cases. To illustrate, consider the collection of information from different viewpoints to reduce the impact of obscurants. As described in the context of FIG. 9, the identification of relatively unobscured views may be performed in real time based on analysis of individual images. However, other approaches may also be possible. For example, in some cases multiple images from different viewpoints may be collected, and, after collection, may be subjected to analysis such as described in the context of FIG. 9 to identify regions of acceptable (e.g., relatively unobscured) visibility in those images. These multiple regions could then be combined using computational approaches (e.g., as described in Xue, T., Rubinstein, M., Liu, C., & Freeman, W. T. (2015), A computational approach for obstruction free photography. ACM Transactions on Graphics (TOG), 34(4), 1-11), the disclosure of which is incorporated by reference herein in its entirety, to create a single unobstructed image such as illustrated in FIG. 10. Similarly, image restoration techniques, such as deblurring and edge enhancement may be used to enhance visibility of critical structures even beyond what would be provided by combining data from multiple images. Further, in cases where there is a known target (e.g., a predefined critical structure such as a nerve or ureter), probabilistic models of the target may be used to augment or bridge any gaps left in images captured by cameras on a surgical device, thereby allowing for clear images to be provided even in cases where a portion of the critical structure may be obscured in all available views.

Variations in data collection and registration of images may also be applied in some cases. For example, as described previously, in some cases position and orientation information gathered during movement of a surgical device may be used directly to register critical structures in multiple images. However, it is also possible that some additional processing may be incorporated into the registration process. For example, in some cases, representations of a critical structure in multiple images may be registered by initially identifying the critical structure in the images (e.g., matching image data with the critical structure's expected position, size and shape as provided by pre-operative images), then treating the registration of the critical structure across images as a rigid transformation. In this approach, the registration between the first and second images can be estimated as an affine transformation modeling translation, rotation, non-isotropic scaling and shear between the critical structure as depicted in the two images, rather than simply relying on tracking of position and orientation information as described above.

As another example of a type of variation which may be implemented, in some cases image processing and combination may be performed for images captured from a single location rather than from multiple viewpoints as described. For example, in some implementations, a surgical device (e.g., an endoscope) may be instrumented with multiple cameras, such as an RGB camera and a molecular chemical imaging (MCI) camera at its tip, and when an image is captured it may be simultaneously captured by both the RGB and MCI cameras. In such a case, data from the MCI camera may be used for detection of a target structure, while data from the RGB camera may be applied to improve the MCI camera's detection. For example, in a case where the disclosed technology is used to detect gallstones, data from the RGB camera can be used to create a glare mask, which can be applied to an initial detection of gallstones made by the MCI camera to identify and remove false positives. This type of false positive identification may also be associated with an initial registration between the images from the RGB and MCI cameras, such as using known positions on the tip of the surgical device (e.g., the distance between the cameras, which will preferably be about 4 mm) or by using rigid transformations like the affine transformation estimation as described above. This type of false positive estimation may also be applied in cases where there are not separate RGB and MCI cameras on a surgical device's tip. For example, in some cases one or more cameras may be provided that could detect both light used in RGB and molecular chemical imaging. When such a camera is available, then a first image may be captured when the surgical site is illuminated with visible light, and a brief time later (e.g., 5 ms, or other time which is short enough to avoid significant distortions caused by breathing or other motion) a second image may be captured when the surgical site is illuminated for molecular chemical imaging.

As another example of a type of variation which may be implemented, in some cases a three dimensional image may be determined based on more than two images. For example, as noted in the discussion of FIG. 9, in some cases images may be captured continuously while a device was being translated and reoriented (902), and/or additional data may be gathered (903) when an informative view was detected during translation and reorientation (902). In some implementations, these additional views, or other images captured during translation and reorientation (903) may be combined into a three dimensional image by registering the critical structure in the additional view(s) with the critical structure in first and second images, normalizing the critical structure in the additional view(s) in a manner similar to that described previously for normalizing (805) the critical structure with a non-rigid transformation in the context of FIG. 8, and then using this normalized and registered critical structure to provide a three dimensional image that takes advantage of information from three or more images, thereby providing a more accurate and/or detailed three dimensional image.

Another example of a type of variation which may be implemented is the use of images which do not share a target structure in common to create a three dimensional image. As noted above, in some instances, a three dimensional image may be created based on three or more underlying images captured of a surgical site. While this may be done by combining images which were all captured when a target structure was within the field of view of a camera, in some cases this may be done by capturing a first image when a first target structure is in the field of view of a camera, a second image when the first target structure and a second target structure is in the field of view of the camera, and a third image when the second target structure is in the field of view of the camera. In this type of scenario, the first and second images may be combined using the first target structure, and the third image may be combined using the second target structure, thereby providing a three dimensional image based on the first, second and third images, despite the fact that the structure used to register the third image was not present in the first image, and the structure used to register the first image was not present in the third image. This approach may also be extended to additional images and target structures (e.g., a fourth image could be integrated by using a third target structure that is shared by the fourth image and at least one other image used to create the three dimensional image), thereby potentially providing for an effective baseline which is significantly larger even than what may be provided by the combination of two images.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method comprising: (a) capturing, using one or more cameras located at a distal tip of a surgical instrument, a set of images, wherein: (i) for each image from the set of images: (A) that image is captured by a corresponding camera from the one or more cameras; and (B) that image is captured when the distal tip of the surgical instrument is located at a corresponding position in space; (ii) the set of images comprises a first image and a second image; (b) generating a three dimensional image based on compositing a representation of a target structure in the first image with a representation of the target structure in the second image, after applying a nonrigid transformation to one or more of: (i) the representation of the target structure in the first image; and (ii) the representation of the target structure in the second image.

### Example 2

The method of Example 1, wherein the method comprises registering the first image and the second image based on applying a rigid transformation to the first image.

### Example 3

The method of Example 2, wherein registering the first image and the second image based on applying the rigid transformation to the first image comprises: (a) identifying a feature which is visible in both the first image and the second image; (b) determining a first transformation mapping the feature in the first image onto the feature in the second image; and (c) applying the first transformation to the first image.

### Example 4

The method of Example 3, wherein determining the first transformation comprises estimating an affine transformation modeling translation, rotation, non-isotropic scaling and shear between the feature in the first image and the second image.

### Example 5

The method of any of Examples 2-4, wherein (a) the method comprises tracking the position and orientation of the surgical instrument in space; and (b) registering the first image and the second image based on applying the rigid transformation to the first image comprises: (i) determining a linear map from the first image to the second image based on: (A) a position and orientation of the surgical instrument when the first image was captured; and (B) a position and orientation of the surgical instrument when the second image was captured; and (ii) applying the linear map to the first image.

### Example 6

The method of any of Examples 2-5, wherein applying the rigid transformation to the first image is performed prior to applying the nonrigid transformation.

### Example 7

The method of any of the preceding Examples, wherein applying the nonrigid transformation to one or more of the representations of the target structure in the first image and the representation of the target structure in the second image comprises interpolating and smoothing the representation of the target structure in the first image with the representation of the target structure in the second image using thin plate splines.

### Example 8

The method of any of Examples 1-6, wherein applying the nonrigid transformation to one or more of the representation of the target structure in the first image and the representation of the target structure in the second image comprises applying a diffusion modeling process to the representation of the target structure in the first image and the representation of the target structure in the second image.

### Example 9

The method of any of the preceding Examples, wherein (a) the corresponding position in space for the first image is different from the corresponding position in space for the second image; and (b) the representation of the target structure in the first image depicts a different portion of the target structure than the representation of the target structure in the second image.

### Example 10

The method of Example 9, wherein (a) the set of images comprises a third image; (b) the corresponding position in space for the third image is different from the corresponding positions in space for the first image and the second image; and (c) the method comprises determining that a portion of the target structure is not represented in any of the first image, the second image and the third image as a result of being blocked by an occlusion.

### Example 11

The method of any of Examples 9-10, wherein the method comprises: (a) determining a position and orientation from which the not represented portion of the target structure can be imaged around the occlusion; and (b) generating an instruction to move the surgical instrument to the determined location and orientation.

### Example 12

The method of Example 11, wherein the method comprises providing the instruction to a user of the surgical instrument.

### Example 13

The method of any of the preceding Examples, wherein the one or more cameras comprises a first camera adapted to capture a RGB image, and a second camera adapted to capture an MCI image.

### Example 14

The method of any of the preceding Examples, wherein (a) the set of images comprises a third image; (b) the corresponding position in space for the third image is different from the corresponding positions in space for the first image and the second image; and (c) generating the three dimensional image comprises compositing a representation of the target structure in the third image with the representation of the target structure in the first image and the representation of the target structure in the second image after applying the nonrigid transformation to the representation of the target structure in the third image.

### Example 15

The method of any of the preceding Examples, wherein the method comprises, prior to compositing the representation of the target structure in the first image with the representation of the target structure in the second image, applying an image enhancement to one or more of: (a) the representation of the target structure in the first image; and (b) the representation of the target structure in the second image.

### Example 16

The method of Example 15, wherein applying the image enhancement comprises: (a) determining a glare mask based on the first image; and (b) applying the glare mask to the second image.

### Example 17

The method of any of the preceding Examples, wherein, for each image from the set of images, the corresponding position in space for that image is inside a body of a patient.

### Example 18

The method of any of Examples 1-16, wherein, for each image from the set of images, the corresponding position in space for that image is inside a body of a patient.

### Example 19

The method of any of the preceding examples, wherein: (a) the set of images comprises a third image; (b) the corresponding position in space for the third image is different from the corresponding positions in space for the first image and the second image; and (c) generating the three dimensional image comprises compositing a representation of a second target structure in the third image with a representation of the second target structure in the first image or the second image after applying the nonrigid transformation to the representation of the target structure in the third image.

### Example 20

A surgical visualization system comprising: (a) a surgical instrument having a distal tip and one or more cameras disposed thereon; (b) a display; (c) a processor; and (d) a memory storing instructions operable to, when executed by the processor, cause performance of a set of acts comprising: (i) capturing, using the one or more cameras located at the distal tip of the surgical instrument, a set of images, wherein: (A) for each image from the set of images: (I) that image is captured by a corresponding camera from the one or more cameras; and (II) that image is captured when the distal tip of the surgical instrument is located at a corresponding position in space; (B) the set of images comprises a first image and a second image; (ii) generating a three dimensional image based on compositing a representation of a target structure in the first image with a representation of the target structure in the second image, after applying a nonrigid transformation to one or more of: (A) the representation of the target structure in the first image; and (B) the representation of the target structure in the second image; and (iii) presenting the three dimensional image to a user on the display.

### Example 21

The surgical visualization system of Example 20, wherein (a) the corresponding position in space for the first image is different from the corresponding position in space for the second image; and (b) the set of acts comprises: (i) identifying a portion of the target structure not obscured by an occlusion in the first image; (ii) identifying a portion of the target structure not obscured by the occlusion in the second image, wherein the portion of the target structure identified as not obscured by the occlusion in the first image is different from the portion of the target structure identified as not obscured by the occlusion in the second image; and (iii) generating the three dimensional image comprises combining the portion of the target structure identified as not obscured by the occlusion in the first image with the portion of the target structure identified as not obscured by the occlusion in the second image.

### Example 22

The surgical visualization system of any of Examples 20 through 21, wherein: (a) the one or more cameras comprises a first camera adapted to capture a RGB image, and a second camera adapted to capture an MCI image; and (b) the set of acts comprises, prior to compositing the representation of the target structure in the first image with the representation of the target structure in the second image: (i) determining a glare mask based on the first image; and (ii) applying the glare mask to the second image.

### Example 23

A non-transitory computer readable medium having stored thereon instructions operable to, when executed by a processor of a surgical visualization system, cause the surgical visualization system to perform acts comprising: (a) capturing, using one or more cameras located at a distal tip of a surgical instrument, a set of images, wherein: (i) for each image from the set of images: (A) that image is captured by a corresponding camera from the one or more cameras; and (B) that image is captured when the distal tip of the surgical instrument is located at a corresponding position in space; (ii) the set of images comprises a first image and a second image; (b) generating a three dimensional image based on compositing a representation of a target structure in the first image with a representation of the target structure in the second image, after applying a nonrigid transformation to one or more of: (i) the representation of the target structure in the first image; and (ii) the representation of the target structure in the second image.

### IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. For instance, while many of the illustrative examples focused in cases in which the disclosed technology was utilized laparoscopically, it is contemplated that the disclosed technology may also be used for open procedures, rather than only being applicable in the laparoscopic context. Various suitable ways in which the teachings herein may be combined will be readily apparent to those of ordinary skill in the art in view of the teachings herein. Such modifications and variations are intended to be included within the scope of the claims.

Furthermore, any one or more of the teachings herein may be combined with any one or more of the teachings disclosed in U.S. Pat. App. No. [Atty. Ref. END9343USNP1], entitled “Endoscope with Source and Pixel Level Image Modulation for Multispectral Imaging,” filed on even date herewith; U.S. Pat. App. No. [Atty. Ref. END9345USNP1], entitled “Scene Adaptive Endoscopic Hyperspectral Imaging System,” filed on even date herewith; and/or U.S. Pat. App. No. [Atty. Ref. END9346USNP1], entitled “Stereoscopic Endoscope with Critical Structure Depth Estimation,” filed on even date herewith. The disclosure of each of these U.S. patent applications is incorporated by reference herein.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they may be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that may penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method comprising:
(a) capturing, using one or more cameras located at a distal tip of a surgical instrument, a set of images, wherein:
(i) for each image from the set of images:
(A) that image is captured by a corresponding camera from the one or more cameras; and
(B) that image is captured when the distal tip of the surgical instrument is located at a corresponding position in space;
(ii) the set of images comprises a first image and a second image; and
(b) generating a three dimensional image based on compositing a representation of a target structure in the first image with a representation of the target structure in the second image, after applying a nonrigid transformation to one or more of:
(i) the representation of the target structure in the first image; and
(ii) the representation of the target structure in the second image.

2. The method of claim 1, wherein the method comprises registering the first image and the second image based on applying a rigid transformation to the first image.

3. The method of claim 2, wherein registering the first image and the second image based on applying the rigid transformation to the first image comprises:
(a) identifying a feature which is visible in both the first image and the second image;
(b) determining a first transformation mapping the feature in the first image onto the feature in the second image; and
(c) applying the first transformation to the first image.

4. The method of claim 3, wherein determining the first transformation comprises estimating an affine transformation modeling translation, rotation, non-isotropic scaling and shear between the feature in the first image and the second image.

5. The method of claim 2, wherein:
(a) the method comprises tracking the position and orientation of the surgical instrument in space; and
(b) registering the first image and the second image based on applying the rigid transformation to the first image comprises:
(i) determining a linear map from the first image to the second image based on:
(A) a position and orientation of the surgical instrument when the first image was captured; and
(B) a position and orientation of the surgical instrument when the second image was captured; and
(ii) applying the linear map to the first image.

6. The method of claim 2, wherein applying the rigid transformation to the first image is performed prior to applying the nonrigid transformation.

7. The method of claim 1, wherein applying the nonrigid transformation to one or more of the representation of the target structure in the first image and the representation of the target structure in the second image comprises interpolating and smoothing the representation of the target structure in the first image with the representation of the target structure in the second image using thin plate splines.

8. The method of claim 1, wherein applying the nonrigid transformation to one or more of the representation of the target structure in the first image and the representation of the target structure in the second image comprises applying a diffusion modeling process to the representation of the target structure in the first image and the representation of the target structure in the second image.

9. The method of claim 1, wherein:
(a) the corresponding position in space for the first image is different from the corresponding position in space for the second image; and
(b) the representation of the target structure in the first image depicts a different portion of the target structure than the representation of the target structure in the second image.

10. The method of claim 9, wherein:
(a) the set of images comprises a third image;
(b) the corresponding position in space for the third image is different from the corresponding positions in space for the first image and the second image; and
(c) the method comprises determining that a portion of the target structure is not represented in any of the first image, the second image and the third image as a result of being blocked by an occlusion.

11. The method of claim 9, wherein the method comprises:
(a) determining a position and orientation from which the not represented portion of the target structure can be imaged around the occlusion; and
(b) generating an instruction to move the surgical instrument to the determined location and orientation.

12. The method of claim 11, wherein the method comprises providing the instruction to a user of the surgical instrument.

13. The method of claim 1, wherein the one or more cameras comprises a first camera adapted to capture a RGB image, and a second camera adapted to capture an MCI image.

14. The method of claim 1, wherein:
(a) the set of images comprises a third image;
(b) the corresponding position in space for the third image is different from the corresponding positions in space for the first image and the second image; and
(c) generating the three dimensional image comprises compositing a representation of the target structure in the third image with the representation of the target structure in the first image and the representation of the target structure in the second image after applying the nonrigid transformation to the representation of the target structure in the third image.

15. The method of claim 1, wherein the method comprises, prior to compositing the representation of the target structure in the first image with the representation of the target structure in the second image, applying an image enhancement to one or more of:
(a) the representation of the target structure in the first image; and
(b) the representation of the target structure in the second image.

16. The method of claim 15, wherein applying the image enhancement comprises:
(a) determining a glare mask based on the first image; and
(b) applying the glare mask to the second image.

17. A surgical visualization system comprising:
(a) a surgical instrument having a distal tip and one or more cameras disposed thereon;
(b) a display;
(c) a processor; and
(d) a memory storing instructions operable to, when executed by the processor, cause performance of a set of acts comprising:
(i) capturing, using the one or more cameras located at the distal tip of the surgical instrument, a set of images, wherein:
(A) for each image from the set of images:
(I) that image is captured by a corresponding camera from the one or more cameras; and
(II) that image is captured when the distal tip of the surgical instrument is located at a corresponding position in space; and
(B) the set of images comprises a first image and a second image;
(ii) generating a three dimensional image based on compositing a representation of a target structure in the first image with a representation of the target structure in the second image, after applying a nonrigid transformation to one or more of:
(A) the representation of the target structure in the first image; and
(B) the representation of the target structure in the second image; and
(iii) presenting the three dimensional image to a user on the display.

18. The system of claim 17, wherein:
(a) the corresponding position in space for the first image is different from the corresponding position in space for the second image; and
(b) the set of acts comprises:
(i) identifying a portion of the target structure not obscured by an occlusion in the first image;
(ii) identifying a portion of the target structure not obscured by the occlusion in the second image, wherein the portion of the target structure identified as not obscured by the occlusion in the first image is different from the portion of the target structure identified as not obscured by the occlusion in the second image; and
(iii) generating the three dimensional image comprises combining the portion of the target structure identified as not obscured by the occlusion in the first image with the portion of the target structure identified as not obscured by the occlusion in the second image.

19. The system of claim 17, wherein:
(a) the one or more cameras comprises a first camera adapted to capture a RGB image, and a second camera adapted to capture an MCI image; and
(b) the set of acts comprises, prior to compositing the representation of the target structure in the first image with the representation of the target structure in the second image:
(i) determining a glare mask based on the first image; and
(ii) applying the glare mask to the second image.

20. A non-transitory computer readable medium having stored thereon instructions operable to, when executed by a processor of a surgical visualization system, cause the surgical visualization system to perform acts comprising:
(a) capturing, using one or more cameras located at a distal tip of a surgical instrument, a set of images, wherein:
(i) for each image from the set of images:
(A) that image is captured by a corresponding camera from the one or more cameras; and
(B) that image is captured when the distal tip of the surgical instrument is located at a corresponding position in space;
(ii) the set of images comprises a first image and a second image;
(b) generating a three dimensional image based on compositing a representation of a target structure in the first image with a representation of the target structure in the second image, after applying a nonrigid transformation to one or more of:
(i) the representation of the target structure in the first image; and
(ii) the representation of the target structure in the second image.
